(19) United States
(12) Patent Application Publication
(10) Pub. No.: US 2015/0139925 A1
(43) Pub. Date: May 21, 2015

(54) COSMETIC COMPOSITION FOR KERATIN FIBERS

(71) Applicants: Yuko Kamikawa, Ibaraki-shi (JP); Daisuke Misu, Yokohama (JP); Maxime De Boni, Tokyo (JP)

(72) Inventors: Yuko Kamikawa, Ibaraki-shi (JP); Daisuke Misu, Yokohama (JP); Maxime De Boni, Tokyo (JP)

(73) Assignee: L'OREAL, Paris (FR)

(21) Appl. No.: 14/390,162

(22) PCT Filed: Apr. 4, 2012
(86) PCT No.: PCT/JP2012/059837

§ 371 Date: Jan. 29, 2015

(51) Int. Cl. A61K 8/04; A61K 8/39; A61K 8/41; A61Q 5/10; A61K 8/44; A61K 8/92; A61K 8/34; A61K 8/67; A61K 8/23; A61Q 5/08; A61K 8/86

(52) U.S. Cl. CPC A61K 8/042; A61Q 5/08; A61K 8/39; A61K 8/416; A61K 8/41; A61Q 5/10; A61K 8/86; A61K 8/92; A61K 8/411; A61K 8/345; A61K 8/676; A61K 8/23; A61K 8/44; A61K 2800/882; A61K 2800/4324; A61K 2800/30
USPC 424/62; 8/406

(57) ABSTRACT

The present invention mainly relates to a cosmetic composition for keratin fibers, comprising: (a) at least one fatty material; (b) at least one nonionic surfactant with an HLB of 10 or less; (c) at least one nonionic surfactant with an HLB of more than 10; and (d) water wherein the weight ratio of the total amount of the (b) nonionic surfactant with an HLB of 10 or less and the (c) nonionic surfactant with an HLB of more than 10 to the amount of the (a) fatty substance is from 0.6 to 6.0, preferably from 0.6 to 5.0, and more preferably from 0.6 to 4.0; and the weight ratio of the total amount of the (a) fatty substance, (b) nonionic surfactant with an HLB of 10 or less and (c) at least one nonionic surfactant with an HLB more than 10 to the amount of the (d) water is from 0.5 to 1.30, preferably from 0.5 to 1.25, and more preferably from 0.5 to 1.20.

### TECHNICAL FIELD

The present invention relates to a cosmetic composition for keratin fibers such as hair.

### BACKGROUND ART

In addition to higher performance of products for bleaching or coloring hair, consumers of such products are more and more sensitive to the usage quality of the products. From the viewpoint of usage quality, for example, malodor from ammonia, which is typically contained as an alkaline agent in conventional hair bleaching or coloring products; difficulty in self-handling conventional hair bleaching or coloring products in the form of a liquid, gel or cream; risk of the product dripping off during application to the hair; and the like can be regarded as major drawbacks.

In order to reduce the problems occurring with the pungent odor of ammonia, total or partial replacement of this alkaline agent with another one, such as monoethanolamine, has been proposed. However, the consequence of this modification is a decrease of the bleaching or coloring efficiency of the composition.

Recently, an alternative to the hair dyeing or bleaching compositions based on ammonia as an alkaline agent has been proposed, with compositions comprising high amounts of fatty materials. Such a composition may comprise more than 40% by weight of fatty compounds, relative to the total weight of the composition, in combination with an oxidizing agent, with or without an oxidative dye. Such compositions provide high bleaching or coloring ability with a relatively small amount of alkaline agent and advantageously without ammonia.

### DISCLOSURE OF INVENTION

However, the above compositions including high amounts of fatty materials can still be improved as for their efficiency, more particularly the homogeneity of the dyeing or of the enlightening. It is also desired to keep the aspect of the composition once it is applied on hair and during the time it is left on hair to act. Indeed, during this time, a change in the consistence of the composition, which hardens, may cause difficulties to spread the composition on hair when necessary (for instance when, near the end of the dyeing process, the composition applied on the root should be spread on the remaining part of the fiber). It is also desired to improve the cosmetic aspect of the hair after the treatment, such as smoothness.

Moreover, no gel-type formulation has been reported for the above composition comprising a relatively high amount of fatty materials without using ammonia, because classical gel-type hair colors have been formulated with hydrophilic solvents such as polyols with a low molecular weight and volatile alcohols that are also known to strongly suppress bleaching capabilities. Especially in the case of using alkanolamines as an alkaline agent, bleaching efficiency of such gel-type hair colors becomes much lower than expectations. Thus, contrary to cream-type hair colors, classical gel-type hair colors have even more disadvantages and until now, no satisfactory gel-type hair color technology has been reported using alkanolamine, especially monoethanolamine.

An objective of the present invention is to provide a cosmetic composition for keratin fibers such as hair which is a gel-type and can realize high bleaching or coloring ability, without using ammonia.

The above objective of the present invention can be achieved by a cosmetic composition for keratin fibers, comprising:

(a) at least one fatty material;

(b) at least one nonionic surfactant with an HLB of 10 or less;

(c) at least one nonionic surfactant with an HLB of more than 10; and

(d) water

wherein

the weight ratio of the total amount of the (b) nonionic surfactant with an HLB of 10 or less and the (c) nonionic surfactant with an HLB of more than 10 to the amount of the (a) fatty substance is from 0.6 to 6.0, preferably from 0.6 to 5.0, and more preferably from 0.6 to 4.0; and

the weight ratio of the total amount of the (a) fatty substance, (b) nonionic surfactant with an HLB of 10 or less and (c) at least one nonionic surfactant with an HLB more than 10 to the amount of the (d) water is from 0.5 to 1.30, preferably from 0.5 to 1.25, and more preferably from 0.5 to 1.20.

It is preferable that the amount of the (a) fatty material(s) be 40% by weight or less, more preferably 30% by weight or less, and even more preferably 20% by weight or less, relative to the total weight of the composition.

The (a) fatty material may be in the form of a liquid or solid.

It is preferable that the (a) fatty material be selected from the group consisting of oils of animal or plant origin, synthetic glycerides, fatty esters other than animal or plant oils and synthetic glycerides, fatty alcohols, fatty acids, silicone oils, and aliphatic hydrocarbons.

It is more preferable that the (a) fatty material be selected from aliphatic hydrocarbons, and particularly is mineral oil.

It is preferable that the (b) nonionic surfactant with an HLB of 10 or less be selected from the group consisting of alcohols, alpha-diols, alkylphenols or esters of fatty acids that are polyethoxylated, polypropoxylated or polyglycerolated and have at least one fatty chain;

copolymers of ethylene oxide and/or of propylene oxide; condensates of ethylene oxide and/or of propylene oxide with fatty alcohols; polyethoxylated fatty amides; polyglycerolated fatty amides; ethoxylated fatty acid esters of sorbitan; ethoxylated oils from plant origin; fatty acid esters of sucrose; fatty acid esters of polyethylene glycol; fatty acid mono or diesters of glycerol; (C₆-C₂₄)alkylpolyglycosides; N—(C₆-C₂₄)alkylglucamine derivatives; and amine oxides.

It is more preferable that the (b) nonionic surfactant with an HLB of 10 or less be selected from polyethoxylated fatty alcohols or esters; the number of ethylene oxide units being between 1 and 6.

It is preferable that the amount of the (b) nonionic surfactant with an HLB of 10 or less be from 1 to 40% by weight, more preferably from 5 to 30% by weight, and even more preferably from 10 to 25% by weight, relative to the total weight of the composition.

It is preferable that the (c) nonionic surfactant with an HLB of more than 10 be selected from the group consisting of alcohols, alpha-diols, alkylphenols or esters of fatty acids that are polyethoxylated, polypropoxylated or polyglycerolated and have at least one fatty chain; copolymers of ethylene oxide and/or of propylene oxide; condensates of ethylene oxide and/or of propylene oxide with fatty alcohols; polyethoxylated fatty amides; polyglycerolated fatty amides; ethoxylated fatty acid esters of sorbitan; ethoxylated oils from plant origin; fatty acid esters of sucrose; fatty acid esters of polyethylene glycol; fatty acid mono or diesters of glycerol; (C₆-C₂₄)alkylpolyglycosides; N—(C₆-C₂₄)alkylglucamine derivatives; and amine oxides.

It is more preferable that the (b) nonionic surfactant with an HLB of 10 or more be selected from polyethoxylated fatty alcohols or esters; the number of ethylene oxide units being between 7 and 100.

It is preferable that the (c) nonionic surfactant with an HLB of more than 10 be from 1 to 30% by weight, more preferably from 3 to 20% by weight, and even more preferably from 5 to 15% by weight, relative to the total weight of the composition.

It is preferable that the amount of the (d) water be from 10 to 70% by weight, more preferably from 20 to 60% by weight, and even more preferably 30 to 50% by weight, relative to the total weight of the composition.

It is preferable that the cosmetic composition according to the present invention further comprise (e) at least one alkaline agent.

It is more preferable that the (e) alkaline agent be selected from non-volatile alkaline agents.

The (e) alkaline agent may be selected from organic amines, inorganic bases, organic amine salts and ammonium salts.

The non-volatile alkaline agent may be an inorganic alkaline agent selected among the alkaline metal hydroxides; alkaline earth metal hydroxides; alkaline metal (hydrogeno)carbonates; alkaline earth metal (hydrogeno) carbonates; ammonium (hydrogeno)carbonates; alkaline metal metasilicates; ammonium metasilicates; and mixtures thereof.

The non-volatile alkaline agent may be an organic alkaline agent selected among monoamines, derivatives and salts thereof; diamines, and derivatives and salts of diamines; polyamines, and derivatives and salts of polyamines; amino acids and derivatives thereof; oligomers of amino acids and derivatives thereof; polymers of amino acids and derivatives thereof; urea and derivatives thereof; guanidine and derivatives thereof; and mixtures thereof.

It is preferable that the non-volatile alkaline agent be selected from alkanolamines, more preferably monoethanolamine and monoisopropanolamine.

The cosmetic composition according to the present invention may further comprise (f) at least one oxidative dye.

The cosmetic composition according to the present invention may be in the form of a gel.

The present invention also relates to a process of dyeing or enlightening keratin fibers in which the cosmetic composition according to the present invention is applied onto said keratin fibers, in the presence of an oxidizing composition comprising at least an oxidizing agent.

In the above process according to the present invention, the cosmetic composition according to the present invention and the oxidizing composition may be mixed prior to the application onto the keratin fibers.

The present invention also relates to a multi-compartment device appropriate to carry out the process according to the present invention, comprising, at least, a first compartment comprising the cosmetic composition according to the present invention, and a second compartment comprising an oxidizing composition.

### BEST MODE FOR CARRYING OUT THE INVENTION

After diligent research, the inventors have discovered that it is possible to provide a cosmetic composition for keratin fibers, such as hair, by combining (a) fatty material, (b) nonionic surfactant with an HLB of 10 or less, (c) nonionic surfactant with an HLB of more than 10, and (d) water, in a specific manner, which can provide the keratin fibers with high bleaching or coloring performance, without using ammonia. Furthermore, the cosmetic composition can comprise a relatively lower amount of fatty material.

Thus, one embodiment of the present invention is a cosmetic composition for keratin fibers, comprising:

(a) at least one fatty material;

(b) at least one nonionic surfactant with an HLB of 10 or less;

(c) at least one nonionic surfactant with an HLB of more than 10; and

(d) water

wherein

the weight ratio of the total amount of the (b) nonionic surfactant with an HLB of 10 or less and the (c) nonionic surfactant with an HLB of more than 10 to the amount of the (a) fatty substance is from 0.6 to 6.0, preferably from 0.6 to 5.0, and more preferably from 0.6 to 4.0; and

the weight ratio of the total amount of the (a) fatty substance, (b) nonionic surfactant with an HLB of 10 or less and (c) at least one nonionic surfactant with an HLB more than 10 to the amount of the (d) water is from 0.5 to 1.30, preferably from 0.5 to 1.25, and more preferably from 0.5 to 1.20.

The cosmetic composition according to the present invention can be in the form of a gel, which is thermodynamically stable and can be used in an alkaline condition. The cosmetic composition may be in the form of a liquid crystal, but can also be an isotropic gel. The cosmetic composition can be transparent.

Hereinafter, the cosmetic composition according to the present invention will be explained in more detail.

(Cosmetic Composition)

(a) Fatty Material

The cosmetic composition according to the present invention comprises at least one (a) fatty material, and two or more fatty materials may be used in combination. Thus, a single type of fatty material or a combination of different types of fatty materials may be used.

The term “fatty material” means an organic compound that is insoluble in water at ordinary temperature (25° C.) and at atmospheric pressure (760 mmHg) (solubility of less than 5%, preferably 1% and even more preferentially 0.1%). The fatty material may contain, in its structure, a sequence of at least two siloxane groups or at least one hydrocarbon-based chain containing at least 6 carbon atoms. In addition, the fatty substances may be soluble in organic solvents under the same temperature and pressure conditions, for instance chloroform, ethanol, benzene or decamethylcyclopentasiloxane.

In the scope of the invention, it has to be noted that the fatty material does not comprise any C₂-C₃ oxyalkylene units or any glycerolated units.

The (a) fatty material may be in the form of a liquid or a solid. Here, “liquid” and “solid” mean that the fatty material is in the form of a liquid or a paste (non-solid) or solid, respectively, at ambient temperature (25° C.) under atmospheric pressure (760 mmHg or 10⁵ Pa). It is preferable that the fatty material be in the form of a liquid or a paste, more preferably in the form of a liquid, at ambient temperature and under atmospheric pressure.

The (a) fatty material may be selected from the group consisting of oils of animal or plant origin, mineral oils, synthetic glycerides, esters of fatty alcohols and/or fatty acids other than animal or plant oils and synthetic glycerides, fatty alcohols, fatty acids, silicone oils and aliphatic hydrocarbons. These fatty materials may be volatile or non-volatile. Preferably, the fatty material is selected from the group consisting of oils of animal or plant origin, synthetic glycerides, fatty esters other than animal or plant oils and synthetic glycerides, fatty alcohols, fatty acids, silicone oils, and aliphatic hydrocarbons. More preferably, the (a) fatty material is selected from aliphatic hydrocarbons, and particularly is mineral oil.

As examples of aliphatic hydrocarbons, mention may be made of, for example, linear or branched hydrocarbons such as mineral oil (e.g., liquid paraffin), paraffin, vaseline or petrolatum, naphthalenes, and the like; hydrogenated polyisobutene, isoeicosan, polydecenes, hydrogenated polyisobutenes such as Parleam, and decene/butene copolymer; and mixtures thereof.

As examples of other aliphatic hydrocarbons, mention may also be made of linear or branched, or possibly cyclic C₆-C₁₆ lower alkanes. Examples that may be mentioned include hexane, undecane, dodecane, tridecane and isoparaffins such as isohexadecane and isodecane.

As example of synthetic glycerides, mention may be made of, for instance, caprylic/capric acid triglycerides, for instance those sold by the company, Stéarineries Dubois or those sold under the names Miglyol® 810, 812 and 818 by the company, Dynamit Nobel.

As examples of silicone oils, mention may be made of, for example, linear organopolysiloxanes such as dimethylpolysiloxanes, methylphenylpolysiloxanes, methylhydrogonpolysiloxanes, and the like; cyclic organopolysiloxanes such as octamethylcyclotetrasiloxane, decamethylcyclopentasiloxane, dodecamethylcyclohexasiloxane, and the like; and mixtures thereof

As examples of plant oils, mention may be made of, for example, linseed oil, camellia oil, macadamia nut oil, sunflower oil, apricot oil, soybean oil, arara oil, hazelnut oil, corn oil, mink oil, olive oil, avocado oil, sasanqua oil, castor oil, safflower oil, jojoba oil, sunflower oil, almond oil, grapeseed oil, sesame oil, soybean oil, peanut oil, and mixtures thereof

As examples of animal oils, mention may be made of, for example, squalene, perhydrosqualene and squalane.

As examples of the esters of a fatty acid and/or of a fatty alcohol, which are advantageously different from the animal or plant oils as well as the synthetic glycerides mentioned above, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

The following may especially be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferable to use ethyl, isopropyl, myristyl, cetyl or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as fatty ester, sugar esters and diesters of C₆-C₃₀ and preferably C₁₂-C₂₂ fatty acids. The term “sugar” means oxygen-bearing hydrocarbon-based compounds containing several alcohol functions, with or without aldehyde or ketone functions, and which contain at least 4 carbon atoms. These sugars may be monosaccharides, oligosaccharides or polysaccharides.

Examples of suitable sugars that may be mentioned include sucrose (or saccharose), glucose, galactose, ribose, fructose, maltose, mannose, arabinose, xylose and lactose, and derivatives thereof, especially alkyl derivatives, such as methyl derivatives, for instance methylglucose.

The sugar esters of fatty acids may be selected especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds.

The esters according to this variant may also be selected from mono-, di-, tri-, tetraesters and polyesters, and mixtures thereof.

These esters may be selected, for example, from oleates, laurates, palmitates, myristates, behenates, cocoates, stearates, linoleates, linolenates, caprates and arachidonates, or mixtures thereof such as, especially, oleo-palmitate, oleo-stearate and palmito-stearate mixed esters.

It is more particularly preferable to use monoesters and diesters and especially sucrose, glucose or methylglucose mono- or dioleates, stearates, behenates, oleopalmitates, linoleates, linolenates and oleostearates.

An example that may be mentioned is the product sold under the name Glucate® DO, which is a methylglucose dioleate, by the company Amerchol.

Examples of esters or mixtures of esters of sugar and of fatty acid that may also be mentioned include:
the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referenced B370 and corresponding to sucrose behenate formed from 20% monoester and 80% di-triester-polyester;
the sucrose mono-dipalmito-stearate sold by the company Goldschmidt under the name Tegosoft® PSE.

The fatty material may be at least one fatty acid, and two or more fatty acids may be used. The fatty acids should be in acidic form (i.e., unsalified, to avoid soaps) and may be saturated or unsaturated and contain from 6 to 30 carbon atoms and in particular from 9 to 30 carbon atoms, which is optionally substituted, in particular with one or more hydroxyl groups (in particular 1 to 4). If they are unsaturated, these compounds may comprise one to three conjugated or non-conjugated carbon-carbon double bonds. They are more particularly selected from myristic acid, palmitic acid, stearic acid, behenic acid, oleic acid, linoleic acid, linolenic acid and isostearic acid. Preferably the fatty material is not a fatty acid.

The fatty material may be at least one fatty alcohol, and two or more fatty alcohols may be used.

The term “fatty alcohol” here means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds.

Among the C₈-C₃₀ fatty alcohols, C₁₂-C₂₂ fatty alcohols, for example, are used. Mention may be made among these of lauryl alcohol, cetyl alcohol, stearyl alcohol, isostearyl alcohol, oleyl alcohol, behenyl alcohol, linoleyl alcohol, undecylenyl alcohol, palmitoleyl alcohol, linolenyl alcohol, myristyl alcohol, arachidonyl alcohol and erucyl alcohol, and mixtures thereof. In one embodiment, cetyl alcohol, stearyl alcohol or a mixture thereof (e.g., cetearyl alcohol), as well as myristyl alcohol, can be used as a solid fatty material. In another embodiment, isostearyl alcohol can be used as a liquid fatty material.

The fatty material may be a wax. Here, “wax” means that the fatty material is substantially in the form of a solid at room temperature (25° C.) under atmospheric pressure (760 mmHg), and has a melting point generally of 35° C. or more. As the waxy fatty material, waxes generally used in cosmetics can be used alone or in combinations thereof.

For example, the wax may be selected from carnauba wax, microcrystalline waxes, ozokerites, hydrogenated jojoba oil, polyethylene waxes such as the wax sold under the name “Performalene 400 Polyethylene” by the company New Phase Technologies, silicone waxes, for instance poly(C₂₄-C₂₈)alkylmethyldimethylsiloxane, such as the product sold under the name “Abil Wax 9810” by the company Goldschmidt, palm butter, the C₂₀-C₄₀ alkyl stearate sold under the name “Kester Wax K82H” by the company Kester Keunen, stearyl benzoate, shellac wax, and mixtures thereof. For example, a wax selected from carnauba wax, candelilla wax, ozokerites, hydrogenated jojoba oil and polyethylene waxes can be used. In at least one embodiment, the wax is preferably selected from candelilla wax and ozokerite, and mixtures thereof.

As the (a) fatty material, mineral oils are in particular preferable.

The amount of the (a) fatty material(s) may be 40% by weight or less, preferably 30% by weight or less, and more preferably 20% by weight or less, relative to the total weight of the cosmetic composition according to the present invention. The amount of the (a) oil(s) may be from 5 to 40% by weight, preferably from 8 to 30% by weight, and more preferably from 10 to 20% by weight, relative to the total weight of the cosmetic composition.

(b) Nonionic Surfactant with HLB of 10 or Less

The cosmetic composition according to the present invention comprises at least one (b) nonionic surfactant with an HLB of 10 or less, and two or more (b) nonionic surfactant with an HLB of 10 or less may be used in combination. Thus, a single type of fatty material or a combination of different types of nonionic surfactant with an HLB of 10 or less may be used.

It should be noted that the HLB value of a mixture of two or more nonionic surfactants is determined by the weight average of the HLB values of the nonionic surfactants.

It is preferable that the (b) nonionic surfactant with an HLB of 10 or less be selected from the group consisting of alcohols, alpha-diols, alkylphenols or esters of fatty acids that are polyethoxylated, polypropoxylated or polyglycerolated and have at least one fatty chain; copolymers of ethylene oxide and/or of propylene oxide; condensates of ethylene oxide and/or of propylene oxide with fatty alcohols; polyethoxylated fatty amides; polyglycerolated fatty amides; ethoxylated fatty acid esters of sorbitan; ethoxylated oils from plant origin; fatty acid esters of sucrose; fatty acid esters of polyethylene glycol; fatty acid mono or diesters of glycerol; (C₆-C₂₄)alkylpolyglycosides; N—(C₆-C₂₄)alkylglucamine derivatives; and amine oxides, with the proviso that they have an HLB value of 10 or less.

It is more preferable that the (b) nonionic surfactant with an HLB of 10 or less be selected from polyethoxylated fatty alcohols or esters; the number of ethylene oxide units being between 1 and 6, preferably between 3 and 6.

For example, Deceth-3 having an HLB value of 9.2 may be used as the (b) nonionic surfactant with an HLB of 10 or less.

The amount of the (b) nonionic surfactant with an HLB of 10 or less may be from 1 to 40% by weight, preferably from 5 to 30% by weight, and more preferably from 10 to 25% by weight, relative to the total weight of the composition.

(c) Nonionic Surfactant with HLB of More than 10

The cosmetic composition according to the present invention comprises at least one (c) nonionic surfactant with an HLB of more than 10, and two or more (c) nonionic surfactant with an HLB of more than 10 may be used in combination. Thus, a single type of fatty material or a combination of different types of nonionic surfactant with an HLB of more than 10 may be used.

It should be noted that the HLB value of a mixture of two or more nonionic surfactants is determined by the weight average of the HLB values of the nonionic surfactants.

It is preferable that the (c) nonionic surfactant with an HLB of more than 10 be selected from the group consisting of alcohols, alpha-diols, alkylphenols or esters of fatty acids that are polyethoxylated, polypropoxylated or polyglycerolated and have at least one fatty chain; copolymers of ethylene oxide and/or of propylene oxide; condensates of ethylene oxide and/or of propylene oxide with fatty alcohols; polyethoxylated fatty amides; polyglycerolated fatty amides; ethoxylated fatty acid esters of sorbitan; ethoxylated oils from plant origin; fatty acid esters of sucrose; fatty acid esters of polyethylene glycol; fatty acid mono or diesters of glycerol; (C₆-C₂₄)alkylpolyglycosides; N—(C₆-C₂₄)alkylglucamine derivatives; and amine oxides, with the proviso that they have an HLB value of more than 10.

It is more preferable that the (c) nonionic surfactant with an HLB of more than 10 be selected from polyethoxylated fatty alcohols or esters; the number of ethylene oxide units being between 7 and 100.

For example, Laureth-12 having an HLB value of 14.5, Oleth-30 having an HLB value of 16.6 and Oleth-10 having an HLB value of 12.4 may be used as the (c) nonionic surfactant with an HLB of more than 10.

The amount of the (c) nonionic surfactant with an HLB of more than 10 may be from 1 to 30% by weight, preferably from 3 to 20% by weight, and more preferably from 5 to 15% by weight, relative to the total weight of the composition.

According to the present invention, the weight ratio of the total amount of the (b) nonionic surfactant with an HLB of 10 or less and the (c) nonionic surfactant with an HLB of more than 10 to the amount of the (a) fatty substance is from 0.6 to 6.0, preferably from 0.6 to 5.0, and more preferably from 0.6 to 4.0.

(d) Water

The cosmetic composition according to the present invention comprises (d) water.

The amount of the (d) water may be from 10 to 70% by weight, preferably from 20 to 60% by weight, and more preferably 30 to 50% by weight, relative to the total weight of the composition.

According to the present invention, the weight ratio of the total amount of the (a) fatty substance, (b) nonionic surfactant with an HLB of 10 or less and (c) at least one nonionic surfactant with an HLB more than 10 to the amount of the (d) water is from 0.5 to 1.30, preferably from 0.5 to 1.25, and more preferably from 0.5 to 1.20.

(e) Alkaline Agent

The cosmetic composition according to the present invention can comprise at least one (e) alkaline agent, and two or more alkaline agents may be used. Thus, a single type of alkaline agent or a combination of different types of alkaline agents may be used.

The (e) alkaline agent is preferably a non-volatile alkaline agent.

The term “non-volatile alkaline agent” means that the alkaline agent does not correspond to ammonia, which is volatile. A non-volatile agent in the scope of the invention means that the vapor pressure is generally lesser than 0.02 mmHg (2.66 Pa) at room temperature.

The non-volatile alkaline agent is preferably selected from organic amines, inorganic bases, organic amine salts and ammonium salts.

The non-volatile alkaline agent may be an inorganic alkaline agent. It is preferable that the inorganic alkaline agent be selected from the group consisting of alkaline metal hydroxides; alkaline earth metal hydroxides; alkaline metal (hydrogeno)carbonates; alkaline earth metal(hydrogeno)carbonates; ammonium (hydrogeno)carbonates; alkaline metal metasilicates; ammonium metasilicates; and mixtures thereof

As examples of the inorganic alkaline agents, mention may be made of sodium hydroxide, potassium hydroxide, calcium hydroxide, magnesium hydroxide, sodium carbonate; potassium carbonate, sodium hydrogenocarbonate, potassium hydrogenocarbonate, and sodium metasilicate.

The non-volatile alkaline agent may be an organic alkaline agent. It is preferable that the organic alkaline agent be selected from the group consisting of monoamines, and derivatives and salts of monoamines, such as alkanolamines; diamines, and derivatives and salts of diamines, such as alkanoldiamines; polyamines, and derivatives and salts of polyamines; amino acids, preferably basic amino acids, and derivatives thereof; oligomers of amino acids, preferably basic amino acids and derivatives thereof; polymers of amino acids, preferably basic amino acids and derivatives thereof; urea and derivatives thereof; guanidine and derivatives thereof; and mixtures thereof

As examples of the organic alkaline agents, mention may be made of alkanolamines such as mono-, di- and tri-ethanolamine, comprising 1 to 3 hydroxyalkyl(C₁-C₄) groups. Particularly, alkanolamines may be selected from monoethanolamine, diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane.

The organic alkaline agents may also be selected from urea, guanidine and their derivatives; amino acids such as alanine, arginine, ornithine, citrulline, asparagine, carnitine, cysteine, glutamine, glycine, histidine, lysine, isoleucine, leucine, methionine, N-phenylalanine, proline, serine, threonine, tryptophan, tyrosine and valine, particularly basic amino acids such as lysine, histidine, ornithine, citrulline or arginine; and diamines such as those described in the structure below:

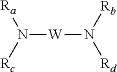

wherein W denotes an alkylene such as propylene optionally substituted by a hydroxyl or a C₁-C₄ alkyl radical, and Rₐ, R_{b}, R_{c} and R_{d} independently denote a hydrogen atom, an alkyl radical or a C₁-C₄ hydroxyalkyl radical, which may be exemplified by 1,3-propanediamine and derivatives thereof. Among amino acids, basic amino acids such as lysine, histidine, ornithine, citrulline or arginine are preferable.

It is preferable that the non-volatile alkaline agent be selected from alkanolamines, and particularly monoethanolamine.

The (e) alkaline agent(s) may be used in a total amount of from 0.01 to 15% by weight, preferably from 0.1 to 10% by weight, and more preferably from 1 to 6% by weight, relative to the total weight of the cosmetic composition according to the present invention.

(f) Oxidative Dye

The cosmetic composition according to the present invention can comprise at least one (f) oxidative dye, and two or more oxidative dyes may be used. Thus, a single type of oxidative dye or a combination of different types of oxidative dye may be used.

The oxidative dye can be selected from oxidation bases, oxidation couplers, and the acid addition salts thereof.

The oxidation base can be selected from those conventionally known in oxidation dyeing, preferably from the group consisting of ortho- and para-phenylenediamines, double bases, ortho- and para-aminophenols, heterocyclic bases and the acid addition salts thereof.

Among the para-phenylenediamines, may be mentioned more particularly para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethylpara-phenylenediamine, N,N-diethyl-para-phenylenediamine, N,N-dipropyl-paraphenylenediamine, 4-amino-N,N-diethyl-3-methylaniline, N,N-bis(β-hydroxyethyl)-paraphenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-paraphenylenediamine, 2-isopropyl-para-phenylenediamine, N-(β-hydroxypropyl)-paraphenylenediamine, 2-hydroxymethyl-para-phenylenediamine, N,N-dimethyl-3-methylpara-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylamino-ethyloxy-para-phenylenediamine, N-(β-methoxyethyl)-para-phenylenediamine, 2-methyl-1-N-β-hydroxyethyl-para-phenylenediamine, N-(4-aminophenyl)-3-hydroxy-pyrrolidine, 2-[{2-[(4-Aminophenyl)amino]ethyl}(2-hydroxyéthyl)amino]-ethanol, and addition salts thereof with an acid. Most particularly preferable bases are para-phenylenediamine, para-tolylenediamine, 2-isopropyl-paraphenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and addition salts thereof with an acid.

Among the double bases, the following bases can be cited: N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4 ‘-amino-3’-methylphenyl)ethylene-diamine, 1,8-bis(2,5-diaminophenoxy)-3,5-dioxaoctane, and addition salts thereof with an acid.

The para-aminophenols that can be used are para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and addition salts thereof with an acid.

The ortho-aminophenols which can be used as oxidation bases in the context of the present invention are selected in particular from 2-aminophenol, 2-amino-1-hydroxy-5-methylbenzene, 2-amino-1-hydroxy-6-methylbenzene, 5-acetamido-2-aminophenol, and addition salts thereof with an acid.

Among the heterocyclic bases which can be used as oxidation bases in the dyeing compositions in accordance with the invention, there may be mentioned more particularly pyridine derivatives, pyrimidine derivatives, pyrazole derivatives, and addition salts thereof with an acid.

Among the pyridine derivatives, there may be mentioned more particularly the compounds described for example in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and addition salts thereof with an acid.

Among the pyrimidine derivatives, there may be mentioned more particularly the compounds described, for example, in Patents DE 2 359 399; JP 88-169571; JP 91-10659 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triamino-pyrimidine, and the pyrazolopyrimidine derivatives such as those mentioned in patent application FR-A-2 750 048 and among which there may be mentioned pyrazolo[1,5-a]pyrimidine-3,7-diamine; 2,5-dimethyl-pyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-amino-pyrazolo[1,5-a]pyrimidin-5-ol; 2-(3-amino-pyrazolo-[1,5-a]pyrimidin-7-ylamino)ethanol, 2-(7-aminopyrazolo[1,5-a]pyrimidin-3-ylamino)ethanol, 2-[(3-amino-pyrazolo[1,5-a]pyrimidin-7-yl)-(2-hydroxy-ethyl)amino]-ethanol, 2-[(7-aminopyrazolo[1,5-a]-pyrimidin-3-yl)-(2-hydroxyethyl)amino]ethanol, 5,6-dimethylpyrazolo-[1,5-a]pyrimidine-3,7-diamine, 2,6-dimethylpyrazolo-[1,5-a]pyrimidine-3,7-diamine, 2,5,N7,N7-tetramethyl-pyrazolo[1,5-a]pyrimidine-3,7-diamine, 3-amino-5-methyl-7-imidazolylpropyl-aminopyrazolo[1,5-a]-pyrimidine, addition salts thereof and their tautomeric forms, when a tautomeric equilibrium exists, and addition salts thereof with an acid.

Among the pyrazole derivatives, there may be mentioned more particularly the compounds described in Patents DE 3 843 892, DE 4 133 957 and patent applications WO 94/08969, WO 94/08970, FR-A-2 733 749 and DE 195 43 988 such as 4,5-diamino-1-methylpyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazino-pyrazole, 1-benzyl-4,5-diamino-3-methyl-pyrazole, 4,5-diamino-3-tert-butyl-1-methylpyrazole, 4,5-diamino-1-tertbutyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxy-methylpyrazole, 4,5-diamino-3-hydroxymethyl-1-methylpyrazole, 4,5-diamino-3-hydroxymethyl-1-isopropyl-pyrazole, 4,5-diamino-3-methyl-1-isopropyl-pyrazole, 4-amino-5-(2′-aminoethyl)amino-1,3-dimethylpyrazole, 3,4,5-triaminopyrazole, 1-methyl-3,4,5-triamino-pyrazole, 3,5-diamino-1-methyl-4-methylaminopyrazole, 3,5-diamino-4-(β-hydroxy-ethyl)amino-1-methylpyrazole, and addition salts thereof with an acid.

Among the heterocyclic bases which can be used as oxidation bases, there may be mentioned more particularly diaminopyrazolopyrazolones and especially 2,3-diamino-6,7-dihydro-1H,5H-[pyrazolo1,2,a]pyrazol-1-one (IV) and the addition salts of these diaminopyrazolopyrazolones with an acid.

The oxidative dye may be an oxidation coupler which can be selected from those conventionally known in oxidation dyeing, preferably from the group consisting of meta-phenylenediamines, meta-aminophenols, meta-diphenols, naphthols, heterocyclic couplers and the acid addition salts thereof.

The heterocyclic couplers may be selected from the group consisting of indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines and addition salts thereof with an acid.

These couplers are more particularly selected from 2,4-diamino-1-(β-hydroxyethyloxy)benzene, 2-methyl-5-aminophenol, 5-N-(β-hydroxyethyl)amino-2-methylphenol, 3-aminophenol, 2-chloro-3-amino-6-methylphenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 2-methyl-5-hydroxyethylaminophenol, 4-amino-2-hydroxytoluene, 1,3-bis(2,4-diaminophenoxy)-propane, sesamol, 1-amino-2-methoxy-4,5-methylene-dioxybenzene, α-naphthol, 6-hydroxyindole, 4-hydroxyindole, 4-hydroxy-N-methylindole, 6-hydroxy-indoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethyl-pyrazolo[3,2-c]-1,2,4-triazole, 2,6-dimethylpyrazolo[1,5-b]-1,2,4-triazole and addition salts thereof with an acid.

In general, the addition acid salts of the oxidation bases and couplers are selected in particular from hydrochlorides, hydrobromides, sulphates, tartrates, lactates and acetates.

The cosmetic composition according to the present invention may comprise (f) oxidative dye(s) in an amount of from 0.0001 to 20% by weight, preferably from 0.0005 to 15% by weight, and more preferably from 0.005 to 10% by weight, relative to the total weight of the composition.

(g) Other Components

The cosmetic composition according to the present invention may comprise at least one (g-1) thickening agent. According to the present invention, two or more thickening agents may be used in combination. Thus, a single type of thickening agent or a combination of different types of thickening agents may be used.

The (g-1) thickening agent may be a rheology modifier selected from hydrophilic or lipophilic, organic or inorganic polymers and non-polymers. Preferably, thickening agents are organic polymers.

The rheology modifier may preferably be selected from cellulose polymers, galactomannans and derivatives thereof, gums of microbial origin, acrylic acid or acrylamidopropanesulfonic acid cross-linked homopolymers; associative polymers; and mixtures thereof.

As the cellulose polymers, for example, mention may be made of:

hydroxyethylcelluloses, hydroxypropylcelluloses, methylcelluloses, ethylhydroxyethylcelluloses, carboxymethylcelluloses, and quaternized cellulose derivatives.

As microbial gums, mention may be made of xanthan gum and scleroglucan gum.

As the acrylic acid or acrylamidopropanesulfonic acid cross-linked homopolymers, for example, mention may be made of:

acrylic acid homopolymers cross-linked with an allylic alcohol ether of the sugar series, for example, the products sold under the names CARBOPOL 980, 981, 954, 2984, and 5984 by the company Goodrich and the products sold under the names SYNTHALEN M and SYNTHALEN

K by the company 3 VSA;

cross-linked acrylamidomethanesulfonic acid homopolymers, cross-linked acrylamidoethanesulfonic acid homopolymers, cross-linked acrylamidopropanesulfonic acid homopolymers, cross-linked 2-acrylamido-2-methylpropanesulfonic acid homopolymers, cross-linked 2-methylacrylamido-2-methylpropanesulfonic acid homopolymers, and cross-linked 2-acrylamido-n-butanesulfonic acid homopolymers, in particular, cross-linked and partially or totally neutralized poly-2-acrylamido-2-methylpropanesulfonic acids are described and prepared, for example, in German Patent No. 196 25 810.

As the associative polymer, any amphiphilic polymer comprising, in its structure, at least one fatty chain and at least one hydrophilic portion, may be used.

The associative polymers in accordance with the present disclosure may be selected from anionic, cationic, nonionic, and amphoteric polymers.

Among the associative anionic polymers that are non-limiting, mention may be made of those comprising at least one hydrophilic unit and at least one fatty-chain allyl ether unit, for example, those in which the at least one hydrophilic unit comprises at least one ethylenic unsaturated anionic monomeric residue selected, for example, from the residue of a vinylcarboxylic acid, the residue of an acrylic acid, and the residue of a methacrylic acid, and in which the at least one fatty-chain allyl ether unit is selected from the residues of the monomers of the formula below:

CH₂═C(R′)CH₂OBₙR

wherein R′ is selected from H and CH₃, B is an ethylenoxy radical, n is 0 or an integer ranging from 1 to 100, and R is selected from hydrocarbon-based radicals selected from alkyl, arylalkyl, aryl, alkylarylm and cycloalkyl radicals comprising from 8 to 30 carbon atoms, for example, from 10 to 24 and further, for example, from 12 to 18 carbon atoms.

Anionic amphiphilic polymers of this type are described and prepared, for example, according to an emulsion polymerization process described in document EP-0 216 479.

Among associative anionic polymers that are non-limiting, mention may be made of include anionic polymers comprising at least one hydrophilic unit of olefinic unsaturated carboxylic acid type, and at least one hydrophobic unit exclusively of (C₁₀-C₃₀) alkyl ester of unsaturated carboxylic acid type.

Further examples include the anionic polymers described and prepared according to U.S. Pat. Nos. 3,915,921 and 4,509,949.

Cationic associative polymers that are non-limiting include quaternized cellulose derivatives and polyacrylates comprising at least one amine side group.

The nonionic associative polymers may be selected from at least one of celluloses modified with groups comprising at least one fatty chain, for example, hydroxyethylcelluloses modified with groups comprising at least one fatty chain, such as alkyl groups, for example, C₈-C₂, arylalkyl and alkylaryl groups, for example, Natrosol Plus Grade 330 CS (C₁₆ alkyls) sold by the company Aqualon,

celluloses modified with polyalkylene glycol alkylphenyl ether groups,

guars, for example, hydroxypropyl guar, modified with groups comprising at least one fatty chain, for example, an alkyl chain,

copolymers of vinylpyrrolidone and of fatty-chain hydrophobic monomeric residues,

copolymers of at least one monomeric residue selected from C₁-C₆ alkyl methacrylates and acrylates, and of amphiphilic monomeric residues comprising at least one fatty chain,

copolymers of monomeric residues selected from hydrophilic methacrylates and acrylates, and of hydrophobic monomeric residues comprising at least one fatty chain, for example, the polyethylene glycol methacrylate/lauryl methacrylate copolymer,

associative polyurethanes, and

mixtures thereof.

For example, the associative polymers may be selected from associative polyurethanes.

In another example, associative polyurethanes may be selected from nonionic block copolymers comprising in the chain both hydrophilic blocks usually of polyoxyethylene nature, and hydrophobic blocks that may be selected from aliphatic sequences, cycloaliphatic sequences, and aromatic sequences.

Further, for example, these polymers may comprise at least two hydrocarbon-based lipophilic chains comprising from 6 to 30 carbon atoms, separated by a hydrophilic block, wherein the hydrocarbon-based lipophilic chains may be selected from pendant chains and chains at the end of a hydrophilic block. In yet another example, the polymers may comprise at least one pendant chain. In another example, the polymers may comprise a hydrocarbon-based chain at one or both ends of a hydrophilic block.

For example, the associative polyurethanes may be blocked in triblock or multiblock form. The hydrophobic blocks may thus be at each end of the chain (for example, a triblock copolymer with a hydrophilic central block) or distributed both at the ends and within the chain (for example a multiblock copolymer). These polymers may also be selected from graft polymers and starburst polymers.

In another example, the associative polyurethanes are triblock copolymers in which the hydrophilic block is a polyoxyethylene chain comprising from 50 to 1000 oxyethylene groups. In general, the associative polyurethanes comprise a urethane bond between the hydrophilic blocks, hence the name.

By way of example, among the associative polymers that may be used, non-limiting mention may be made of the polymer C₁₆-OE₁₂₀-C₁₆ from the company Servo Delden (under the name SER AD FX1100, which comprises a urethane functional group and has a weight-average molecular weight of 1300), wherein OE is an oxyethylene unit. In another example, an associative polymer that may also be used is Rheolate 205 comprising a urea functional group, sold by the company Rheox, or Rheolate 208 or 204. These associative polyurethanes are sold in pure form.

In yet another example, the product DW 1206B from Rohm & Haas comprising a C₂₀ alkyl chain with a urethane bond, sold at a solids content of 20% in water, may also be used.

Further, for example, it is also possible to use solutions or dispersions of these polymers, for example, in water or an aqueous-alcoholic medium. Examples of such polymers include SER AD FX1010, SER AD FX1035 and SER AD 1070 from the company Servo Delden, and Rheolate 255, Rheolate 278, and Rheolate 244 sold by the company Rheox. It is also possible to use the product DW 1206F and DW 1206J, and also Acrysol RM 184 or Acrysol 44 from the company Rohm & Haas, or alternatively Borchigel LW 44 from the company Borchers.

In yet another example, the polymers that may be used include those described in the article by G Fonnum, J. Bakke and Fk. Hansen—Colloid Polym. Sci 271, 380-389 (1993).

The (g-1) thickening agent may more preferably be selected from cellulose polymers or microbial gums.

According to one embodiment of the present invention, the amount of the (g-1) thickening agent(s), preferably organic thickening polymers, may range from 0.05 to 20% by weight, preferably from 0.1 to 15% by weight, and more preferably from 0.5 to 10% by weight, relative to the total weight of the cosmetic composition according to the present invention.

The cosmetic composition according to the present invention may comprise at least one selected from the group consisting of (g-2) anionic surfactants, (g-3) amophoteric surfactants and (g-4) cationic surfactants.

(g-2) Anionic Surfactants

According to the present invention, the type of anionic surfactant is not limited. It is preferable that the anionic surfactant be selected from the group consisting of (C₆-C₃₀)alkyl sulfates, (C₆-C₃₀)alkyl ether sulfates, (C₆-C₃₀)alkylamido ether sulfates, alkylaryl polyether sulfates, monoglyceride sulfates; (C₆-C₃₀)alkylsulfonates, (C₆-C₃₀)alkylamide sulfonates, (C₆-C₃₀)alkylaryl sulfonates, α-olefin sulfonates, paraffin sulfonates; (C₆-C₃₀)alkyl phosphates; (C₆-C₃₀)alkyl sulfosuccinates, (C₆-C₃₀)alkyl ether sulfosuccinates, (C₆-C₃₀)alkylamide sulfosuccinates; (C₆-C₃₀)alkyl sulfoacetates; (C₆-C₂₄)acyl sarcosinates; (C₆-C₂₄)acyl glutamates; (C₆-C₃₀)alkylpolyglycoside carboxylic ethers; (C₆-C₃₀)alkylpolyglycoside sulfosuccinates; (C₆-C₃₀)alkyl sulfosuccinamates; (C₆-C₂₄)acyl isethionates; N—(C₆-C₂₄)acyl taurates; C₆-C₃₀ fatty acid salts; coconut oil acid salts or hydrogenated coconut oil acid salts; (C₈-C₂₀)acyl lactylates; (C₆-C₃₀)alkyl-D-galactoside uronic acid salts; polyoxyalkylenated (C₆-C₃₀)alkyl ether carboxylic acid salts; polyoxyalkylenated (C₆-C₃₀)alkylaryl ether carboxylic acid salts; and polyoxyalkylenated (C₆-C₃₀)alkylamido ether carboxylic acid salts.

It is more preferable that the anionic surfactant be selected from salts of (C₆-C₃₀)alkyl sulfate or polyoxyalkylenated (C₆-C₃₀)alkyl ether carboxylic acid salts.

In at least one embodiment, the anionic surfactants are in the form of salts such as salts of alkali metals, for instance sodium; salts of alkaline-earth metals, for instance magnesium; ammonium salts; amine salts; and amino alcohol salts. Depending on the conditions, they may also be in acid form.

(g-3) Amphoteric Surfactants

According to the present invention, the type of amphoteric surfactant is not limited. The amphoteric or zwitterionic surfactants can be, for example (non-limiting list), amine derivatives such as aliphatic secondary or tertiary amines, and optionally quaternized amine derivatives, in which the aliphatic radical is a linear or branched chain comprising 8 to 22 carbon atoms and containing at least one water-solubilizing anionic group (for example, carboxylate, sulphonate, sulphate, phosphate or phosphonate).

The amphoteric surfactant may preferably be selected from the group consisting of betaines and amidoaminecarboxylated derivatives.

The betaine-type amphoteric surfactant is preferably selected from the group consisting of alkylbetaines, alkylamidoalkylbetaines, sulfobetaines, phosphobetaines, and alkylamidoalkylsulfobetaines, in particular, (C₈-C₂₄)alkylbetaines, (C₈-C₂₄)alkylamido(C₁-C₈)alkylbetaines, sulphobetaines, and (C₈-C₂₄)alkylamido(C₁-C₈)alkylsulphobetaines. In one embodiment, the amphoteric surfactants of betaine type are selected from (C₈-C₂₄)alkylbetaines, (C₈-C₂₄)alkylamido(C₁-C₈)alkylsulphobetaines, sulphobetaines, and phosphobetaines.

Non-limiting examples that may be mentioned include the compounds classified in the CTFA dictionary, 9th edition, 2002, under the names cocobetaine, laurylbetaine, cetylbetaine, coco/oleamidopropylbetaine, cocamidopropylbetaine, palmitamidopropylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

R₁—CONHCH₂CH₂—N⁺(R₂)(R₃)(CH₂COO⁻)

in which:

R₁ denotes an alkyl radical of an acid. R₁—COOH present in hydrolysed coconut oil, a heptyl, nonyl or undecyl radical,

R₂ denotes a beta-hydroxyethyl group, and

R₃ denotes a carboxymethyl group; and

R₁′—CONHCH₂CH₂—N(B)(C)

in which:

B represents —CH₂CH₂OX′,

C represents —(CH₂)_{z}—Y′, with z=1 or 2,

X′ denotes a —CH₂CH₂—COOH group, —CH₂—COOZ′, —CH₂CH₂—COOH, —CH₂CH₂—COOZ′ or a hydrogen atom,

Y′ denotes —COOH, —COOZ′, —CH₂—CHOH—SO₃Z′ or a —CH₂—CHOH—SO₃H radical,

Z′ represents an ion of an alkaline or alkaline earth metal such as sodium, an ammonium ion or an ion issued from an organic amine, and

R₁′ denotes an alkyl radical of an acid R₁′-COOH present in coconut oil or in hydrolysed linseed oil, an alkyl radical, such as a C₇, C₉, C₁₁ or C₁₃ alkyl radical, a C₁₇ alkyl radical and its iso form, or an unsaturated C₁₇ radical.

It is preferable that the amphoteric surfactant be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

(g-4) Cationic Surfactants

According to the present invention, the type of cationic surfactant is not limited. The cationic surfactant may be selected from the group consisting of optionally polyoxyalkylenated, primary, secondary or tertiary fatty amine salts, quaternary ammonium salts, and mixtures thereof.

Examples of quaternary ammonium salts that may be mentioned include, but are not limited to: those of general formula (I) below:

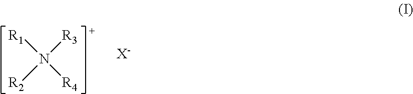

wherein

R₁, R₂, R₃, and R₄, which may be identical or different, are selected from linear and branched aliphatic radicals comprising from 1 to 30 carbon atoms and optionally comprising heteroatoms such as oxygen, nitrogen, sulfur and halogens. The aliphatic radicals may be selected, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals; and aromatic radicals such as aryl and alkylaryl; and X⁻ is selected from halides, phosphates, acetates, lactates, (C₂-C₆) alkyl sulfates and alkyl- or alkylaryl-sulfonates;

quaternary ammonium salts of imidazoline, for instance those of formula (II) below:

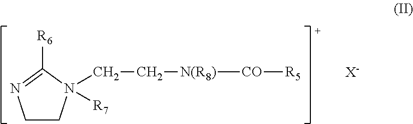

wherein:

R₅ is selected from alkenyl and alkyl radicals comprising from 8 to 30 carbon atoms, for example fatty acid derivatives of tallow or of coconut;

R₆ is selected from hydrogen, C₁-C₄ alkyl radicals, and alkenyl and alkyl radicals comprising from 8 to 30 carbon atoms;

R₇ is selected from C₁-C₄ alkyl radicals;

R₈ is selected from hydrogen and C₁-C₄ alkyl radicals; and

X⁻ is selected from halides, phosphates, acetates, lactates, alkyl sulfates, alkyl sulfonates, and alkylaryl sulfonates. In one embodiment, R₅ and R₆ are, for example, a mixture of radicals selected from alkenyl and alkyl radicals comprising from 12 to 21 carbon atoms, such as fatty acid derivatives of tallow, R₇ is methyl and R₈ is hydrogen. Examples of such products include, but are not limited to, Quaternium-27 (CTFA 1997) and Quaternium-83 (CTFA 1997), which are sold under the names “Rewoquat®” W75, W90, W75PG and W75HPG by the company Witco; diquaternary ammonium salts of formula (III):

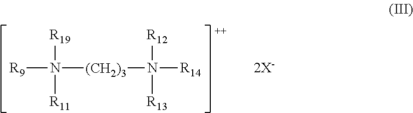

wherein:

R₉ is selected from aliphatic radicals comprising from 16 to 30 carbon atoms;

R₁₀ is selected from hydrogen or alkyl radicals comprising from 1 to 4 carbon atoms or a group (R₁₆ₐ)(R₁₇ₐ(R₁₈ₐ)N⁺(CH₂)₃;

R₁₁, R₁₂, R₁₃, R₁₄, R₁₆ₐ, R₁₇ₐ, and R₁₈ₐ, which may be identical or different, are selected from hydrogen and alkyl radicals comprising from 1 to 4 carbon atoms; and

X⁻ is selected from halides, acetates, phosphates, nitrates, ethyl sulfates, and methyl sulfates.

An example of one such diquaternary ammonium salt is FINQUAT CTP of FINETEX (Quaternium-89) or FINQUAT CT of FINETEX (Quaternium-75); and quaternary ammonium salts comprising at least one ester function, such as those of formula (IV) below:

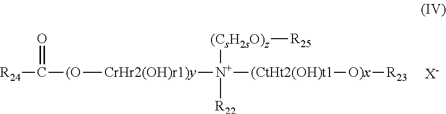

wherein:

R₂₂ is selected from C₁-C₆ alkyl radicals and C₁-C₆ hydroxyalkyl and dihydroxyalkyl radicals;

R₂₃ is selected from:

the radical blow:

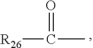

linear and branched, saturated and unsaturated C₁-C₂₂ hydrocarbon-based radicals R₂₇, and

hydrogen,

R₂₅ is selected from:

the radical below:

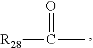

linear and branched, saturated and unsaturated C₁-C₆ hydrocarbon-based radicals R₂₉, and hydrogen,

R₂₄, R₂₆, and R₂₈, which may be identical or different, are selected from linear and branched, saturated and unsaturated, C₇-C₂₁, hydrocarbon-based radicals;

r, s, and t, which may be identical or different, are selected from integers ranging from 2 to 6;

each of r1 and t1, which may be identical or different, is 0 or 1, and r2+r1=2r and t1+2t=2t;

y is selected from integers ranging from 1 to 10;

x and z, which may be identical or different, are selected from integers ranging from 0 to 10;

X⁻ is selected from simple and complex, organic and inorganic anions; with the proviso that the sum x+y+z ranges from 1 to 15, that when x is 0, R₂₃ denotes R₂₇, and that when z is 0, R₂₅ denotes R₂₉. R₂₂ may be selected from linear and branched alkyl radicals. In one embodiment, R₂₂ is selected from linear alkyl radicals. In another embodiment, R₂₂ is selected from methyl, ethyl, hydroxyethyl, and dihydroxypropyl radicals, for example methyl and ethyl radicals. In one embodiment, the sum x+y+z ranges from 1 to 10. When R₂₃ is a hydrocarbon-based radical R₂₇, it may be long and comprise from 12 to 22 carbon atoms, or short and comprise from 1 to 3 carbon atoms. When R₂₅ is a hydrocarbon-based radical R₂₉, it may comprise, for example, from 1 to 3 carbon atoms. By way of a non-limiting example, in one embodiment, R₂₄, R₂₆, and R₂₈, which may be identical or different, are selected from linear and branched, saturated and unsaturated, C₁₁-C₂₁ hydrocarbon-based radicals, for example from linear and branched, saturated and unsaturated C₁₁-C₂₁ alkyl and alkenyl radicals. In another embodiment, x and z, which may be identical or different, are 0 or 1. In one embodiment, y is equal to 1. In another embodiment, r, s and t, which may be identical or different, are equal to 2 or 3, for example equal to 2. The anion X⁻ may be selected from, for example, halides, such as chloride, bromide, and iodide; and C₁-C₄ alkyl sulfates, such as methyl sulfate. However, methanesulfonate, phosphate, nitrate, tosylate, an anion derived from an organic acid, such as acetate and lactate, and any other anion that is compatible with the ammonium comprising an ester function, are other non-limiting examples of anions that may be used according to the invention. In one embodiment, the anion X⁻ is selected from chloride and methyl sulfate.

In another embodiment, the ammonium salts of formula (IV) may be used, wherein:

R₂₂ is selected from methyl and ethyl radicals,

x and y are equal to 1;

z is equal to 0 or 1;

r, s and t are equal to 2;

R₂₃ is selected from:

the radical below:

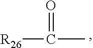

methyl, ethyl, and C₁₄-C₂₂ hydrocarbon-based radicals, hydrogen;

R₂₅ is selected from:

the radical below:

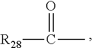

and hydrogen;

R₂₄, R₂₆, and R₂₈, which may be identical or different, are selected from linear and branched, saturated and unsaturated, C₁₃-C₁₇ hydrocarbon-based radicals, for example from linear and branched, saturated and unsaturated, C₁₃-C₁₇ alkyl and alkenyl radicals.

In one embodiment, the hydrocarbon-based radicals are linear.

Non-limiting examples of compounds of formula (IV) that may be mentioned include salts, for example chloride and methyl sulfate, of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethyl-ammonium, and mixtures thereof. In one embodiment, the acyl radicals may comprise from 14 to 18 carbon atoms, and may be derived, for example, from a plant oil, for instance palm oil and sunflower oil. When the compound comprises several acyl radicals, these radicals may be identical or different.

These products may be obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification may be followed by a quaternization using an alkylating agent selected from alkyl halides, for example methyl and ethyl halides; dialkyl sulfates, for example dimethyl and diethyl sulfates; methyl methanesulfonate; methyl para-toluenesulfonate; glycol chlorohydrin; and glycerol chlorohydrin.

Such compounds are sold, for example, under the names Dehyquart® by the company Cognis, Stepanquat® by the company Stepan, Noxamium® by the company Ceca, and “Rewoquat® WE 18” by the company Rewo-Goldschmidt.

Other non-limiting examples of ammonium salts that may be used in the compositions according to the invention include the ammonium salts comprising at least one ester function described in U.S. Pat. Nos. 4,874,554 and 4,137,180.

Among the quaternary ammonium salts mentioned above that may be used in compositions according to the invention include, but are not limited to, those corresponding to formula (I), for example tetraalkylammonium chlorides, for instance dialkyldimethylammonium and alkyltrimethylammonium chlorides in which the alkyl radical comprises from about 12 to 22 carbon atoms, such as behenyltrimethylammonium, distearyldimethylammonium, cetyltrimethylammonium and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the cationic surfactant that may be used in the compositions of the invention is selected from quaternary ammonium salts, for example from behenyltrimethylammonium chloride, cetyltrimethylammonium chloride, Quaternium-83, Quaternium-87, Quaternium-22, behenylamidopropyl-2,3-dihydroxypropyldimethylammonium chloride, palmitylamidopropyltrimethylammonium chloride, and stearamidopropyldimethylamine.

The cosmetic composition according to the present invention may also comprise an effective amount of other agents, known previously elsewhere in lightening or coloring compositions, such as various common adjuvants, sequestering agents such as EDTA and etidronic acid, UV screening agents, silicones other than those mentioned before such as organomodified silicones (such as with amine groups), preserving agents, vitamins or provitamins, for instance, panthenol, opacifiers, fragrances, plant extracts, cationic polymers and so on.

The cosmetic composition according to the present invention may further comprise at least one organic solvent. So the organic solvent is preferably water miscible. As the organic solvent, there may be mentioned, for example, C₁-C₄ alkanols, such as ethanol and isopropanol; polyols and polyol ethers such as glycerol, 2-butoxyethanol, propylene glycol, monomethyl ether of propylene glycol, monoethyl ether, and monomethyl ether of diethylene glycol; and aromatic alcohols such as benzyl alcohol and phenoxyethanol; analogous products; and mixtures thereof.

The organic water-soluble solvents may be present in an amount ranging from less than 10% by weight, preferably from 5% by weight or less, and more preferably from 1% by weight or less, relative to the total weight of the composition. It is preferable that the cosmetic composition according to the present invention does not contain any hydrophilic organic solvent such as polyols with a low molecular weight and volatile alcohols.

The pH of the cosmetic composition according to the present invention may be generally, for example, from 4 to 12. It can range from 6 to 12, preferably 7 to 11, and may be adjusted to the desired value using at least one acidifying agent that is well known in the prior art.

The acidifying agents can be, for example, mineral or organic acids, for instance, hydrochloric acid and orthophosphoric acid, carboxylic acids, for instance, tartaric acid, citric acid, and lactic acid, or sulphonic acids.

The viscosity of the cosmetic composition according to the present invention is not particularly limited. The viscosity can be measured at 25° C. with viscosimeters or rheometers preferably with coneplan geometry. Preferably, the viscosity of the cosmetic composition according to the present invention can range, for example, from 1 to 2000 Pa·s, and preferably from 1 to 1000 Pa·s at 25° C. and 1 s¹.

The composition according to the invention may also comprise a propellant. For the purposes of the present invention, the term “propellant” means any compound that is gaseous at a temperature of 20° C. and at atmospheric pressure, and that can be stored under pressure in liquid or gaseous form in an aerosol container.

The propellant may be selected from optionally halogenated volatile hydrocarbons, such as n-butane, propane, isobutane, pentane, or a halogenated hydrocarbon, and mixtures thereof. Carbon dioxide, nitrous oxide, dimethyl ether (DME), nitrogen, or compressed air may also be used as the propellant. Mixtures of propellants may also be used. Dimethyl ether and/or non-halogenated volatile hydrocarbons are preferably used.

The propellant may be present in the composition in a content of from 1 to 15% by weight, preferably from 2 to 10% by weight, and more preferably from 3 to 8% by weight, relative to the total weight of the composition.

The cosmetic composition according to the present invention can be prepared by mixing at least the essential components (a) to (d), together with the above optional component(s), if necessary, typically the components (e) and/or (f).

(Cosmetic Process)

The present invention also relates to a cosmetic process of dyeing or enlightening (e.g., bleaching) keratin fibers such as hair, comprising applying the cosmetic composition as described above to the keratin fibers, in the presence of an oxidizing composition comprising at least one oxidizing agent.

It is preferable that the total amount of the (a) fatty material(s) be 25% by weight or less, preferably 20% by weight or less, and more preferably 15% by weight or less, relative to the total weight of the compositions.

As the oxidizing agent, a single type of oxidizing agent or a combination of different types of oxidizing agents may be used.

The oxidizing agent may be selected from hydrogen peroxide, peroxygenated salts, and compounds capable of producing hydrogen peroxide by hydrolysis. For example, the oxidizing agent can be selected from an aqueous hydrogen peroxide solution, urea peroxide, alkali metal bromates, and ferricyanides, and persalts such as perborates and persulphates.

It is preferable that the oxidizing agent be hydrogen peroxide.

The oxidizing agent's concentration may range from 0.1 to 15% by weight, preferably from 0.5 to 10% by weight, and more preferably 1 to 5% by weight, relative to the total weight of the oxidizing composition.

In one embodiment, when the oxidizing agent is hydrogen peroxide, the oxidizing composition may comprise at least one hydrogen peroxide stabilizer, which may be selected, for example, from alkali metal and alkaline-earth metal pyrophosphates, alkali metal and alkaline-earth metal stannates, phenacetin, and salts of acids and of oxyquinoline, for example, oxyquinoline sulphate. In another embodiment, at least one stannate optionally in combination with at least one pyrophosphate is used.

It is also possible to use salicylic acid and salts thereof, pyridinedicarboxylic acid and its salts, and paracetamol.

Moreover, the concentration of the hydrogen peroxide stabilizer may range from 0.0001 to 5% by weight such as from 0.01 to 2% by weight, relative to the total weight of the oxidizing composition.

In the composition comprising hydrogen peroxide, the concentration ratio of the hydrogen peroxide to the stabilizer may range from 0.05:1 to 1,000:1, such as from 0.1:1 to 500:1, and further such as from 1:1 to 300:1.

The oxidizing composition may further comprise any of the optional components described above for the cosmetic composition according to the present invention.

In a preferable embodiment, the cosmetic composition according to the invention is mixed prior to being applied on hair, with an oxidizing composition.

Thus, a process according to the invention can comprise the following steps:

applying the cosmetic composition according to the present invention, in the presence of the oxidizing composition, on wet or dry keratin fibers;

keeping the mixture of the cosmetic composition and the oxidizing composition on the fibers for approximately 1 to 60 minutes, or approximately 5 to 45 minutes;

rinsing the fibers; and

optionally washing them with shampoo, rinsing them again and then drying them.

The application of the cosmetic composition according to the present invention or the mixture of the cosmetic composition according to the present invention and the oxidizing composition may be realized at room temperature or with the use of a warming device which is able to produce a temperature ranging from 40 to 220° C., preferably ranging from 40 to 80° C.

(Multi-Compartment Device)

The present invention also relates to a multi-compartment device.

The cosmetic composition according to the present invention may be formulated into a multi-compartment device or kit comprising at least a first compartment and a second compartment in which the first compartment comprises the cosmetic composition according to the present invention, and the second compartment comprises an oxidizing composition.

It is preferable that the amount of the (a) fatty materials in the mixture obtained by mixing the compositions in the first and second compartments be 25% by weight or less, preferably 20% by weight or less, and more preferably 15% by weight or less, relative to the total weight of the mixture.

The multi-compartment device may be equipped with a means for mixing and/or applying the above compositions, such as a valve and a nozzle.

### EXAMPLES

The present invention will be described in more detail by way of examples, which however should not be construed as limiting the scope of the present invention.

### Examples 1-2 and Comparative Examples 1-3

The following compositions according to Examples 1-2 and Comparative Examples 1-3, shown in Table 1, were prepared by mixing the components shown in Table 1. The numerical values for the amounts of the components shown in the Tables are all based on “% by weight” as active raw materials.

TABLE 1

| | | | Comp. | Comp. | Comp. |
|---|---|---|---|---|---|
| Ingredient | Ex. 1 | Ex. 2 | Ex. 1 | Ex. 2 | Ex. 3 |
| Mineral Oil | 15.00 | 15.00 | 5.00 | 15.00 | 15.00 |
| Deceth-3 (90%) | 20.00 | 20.00 | 20.00 | 5.00 | 18.70 |
| Laureth-12 | 4.25 | 4.25 | 4.25 | 1.00 | 4.25 |
| Oleth-30 | 4.25 | 4.25 | 4.25 | 1.00 | 4.25 |
| Oleth-10 | 3.00 | 3.00 | 3.00 | 0.75 | 3.00 |
| Cetrimonium Chloride | 4.00 | 4.00 | 4.00 | 1.00 | 4.00 |
| (25%) | | | | | |
| Ethanolamine | 5.00 | 5.00 | 5.00 | 5.00 | 5.00 |
| Propylene Glycol | — | — | — | — | 10.00 |
| p-Phenylenediamine | — | 0.50 | — | — | — |
| p-Aminophenol | — | 0.50 | — | — | — |
| 4-Amino-2- | — | 0.50 | — | — | — |
| hydroxytoluene | | | | | |
| Resorcinol | — | 0.50 | — | — | — |
| Ascorbic Acid | 0.50 | 0.50 | 0.50 | 0.50 | 0.50 |
| Sodium Metabaisulfite | 0.50 | 0.50 | 0.50 | 0.50 | 0.50 |
| Pentasodium Pentetate | 0.50 | 0.50 | 0.50 | 0.50 | 0.50 |
| (40%) | | | | | |
| Water | Balance | Balance | Balance | Balance | Balance |

[Bleaching Ability]

Each formulation of Examples 1-2 and Comparative Examples 1-3 was mixed with a developer containing 6% hydrogen peroxide in a weight ratio of 1:1.5 (formulation:developer). 30 g of the obtained mixture was applied to 10 g of a lock of natural Japanese black hair for 30 minutes at 27° C.

The hair lock was then shampooed. After being dried in the open air, ΔE (between the color of the original hair and the color of the bleached hair according to the L*a*b* system) was determined with a colorimeter (Konica-Minolta: CM-3600d) by comparing the observed values of the treated hair with those of the untreated black hair. Three experiments were repeated and the results are shown as the average of these data.

The bleaching efficiency was determined in accordance with the following criteria. The results are shown in Table 2.

| Range of ΔE | Bleaching ability |
|---|---|
| Less than 5 | Low |
| Between 5-7 | Medium |
| More than 8 | High |

[Appearance and Phase Structure]

Formulations of Example 1 and Comparative Examples 1-3 were evaluated based on naked eye-observation, and were classified into the following 3 categories: white/translucent/transparent.

The formulations of Example 1 and Comparative Examples 1-3 were studied under an OLYMPUS BX51 optical photomicroscope to determine the phase structures, and they were classified into the following 3 categories: isotropic gel (solubilization)/emulsion/liquid-crystal

The results are shown in Table 2.

TABLE 2

| | | Comp. | Comp. | Comp. |
|---|---|---|---|---|
| | Ex. 1 | Ex. 1 | Ex. 2 | Ex. 3 |
| Bleaching Ability | High | Medium | Medium | Medium |
| Aspect | Transparent | White | White | Translucent |
| Phase Structure | Isotropic gel | Emulsion | Emulsion | Liquid crystal |

Only the formulation of Example 1 was able to satisfy both high bleaching ability and transparent gel aspect.

[Coloring Property]

The formulation of Example 2 was applied on a sample of Japanese natural hair, Japanese bleached hair (light brown), and white goat fur in a weight ratio of 3:1 (3 for weight of the formulation of Example 2 and 1 for weight of the hair or goat fur). The applied hair sample and goat fur were then left for 30 minutes at 27° C. followed by shampooing and drying in the air. The color of the hair samples and goat fur was checked by direct naked-eye observation under controlled daylight conditions. It was found that Example 2 produced a reddish brown color on the goat fur and on the bleached hair. On Japanese natural hair, a dark warm brown color was obtained.

1-23. (canceled)

24. A cosmetic composition for keratin fibers, comprising:
(a) at least one fatty substance;
(b) at least one nonionic surfactant with an HLB of 10 or less;
(c) at least one nonionic surfactant with an HLB of greater than 10; and
(d) water
wherein
the weight ratio of the total amount of the (b) at least one nonionic surfactant with an HLB of 10 or less and the (c) at least one nonionic surfactant with an HLB of greater than 10, to the amount of the (a) at least one fatty substance ranges from 0.6 to 6.0, and
the weight ratio of the total amount of the (a) at least one fatty substance, (b) at least one nonionic surfactant with an HLB of 10 or less, and (c) at least one nonionic surfactant with an HLB greater than 10, to the amount of the (d) water ranges from 0.5 to 1.30.

25. The cosmetic composition according to claim 24, wherein the amount of the (a) at least one fatty substance is about 40% by weight or less, relative to the total weight of the composition.

26. The cosmetic composition according to claim 24, wherein the (a) at least one fatty substance is in the form of a liquid or solid.

27. The cosmetic composition according to claim 26, wherein the (a) at least one fatty substance is chosen from oils of animal or plant origin, synthetic glycerides, fatty esters other than animal or plant oils and synthetic glycerides, fatty alcohols, fatty acids, silicone oils, and aliphatic hydrocarbons, and mixtures thereof.

28. The cosmetic composition according to claim 27, wherein the (a) at least one fatty substance is chosen from aliphatic hydrocarbons.

29. The cosmetic composition according to claim 24, wherein the (b) at least one nonionic surfactant with an HLB of 10 or less is chosen from alcohols, alpha-diols, alkylphenols or esters of fatty acids that are polyethoxylated, polypropoxylated or polyglycerolated and have at least one fatty chain; copolymers of ethylene oxide and/or of propylene oxide; condensates of ethylene oxide and/or of propylene oxide with fatty alcohols; polyethoxylated fatty amides; polyglycerolated fatty amides; ethoxylated fatty acid esters of sorbitan; ethoxylated oils from plant origin; fatty acid esters of sucrose; fatty acid esters of polyethylene glycol; fatty acid mono or diesters of glycerol; (C₆-C₂₄)alkylpolyglycosides; N—(C₆-C₂₄)alkylglucamine derivatives; and amine oxides, and mixtures thereof.

30. The cosmetic composition according to claim 29, wherein the (b) at least one nonionic surfactant with an HLB of 10 or less is chosen from polyethoxylated fatty alcohols or esters, wherein the number of ethylene oxide units ranges from 1 to 6.

31. The cosmetic composition according to claim 24, wherein the amount of the (b) at least one nonionic surfactant with an HLB of 10 or less ranges from about 1% to about 40% by weight, relative to the total weight of the composition.

32. The cosmetic composition according to claim 24, wherein the (c) at least one nonionic surfactant with an HLB of greater than 10 is chosen from alcohols, alpha-diols, alkylphenols or esters of fatty acids that are polyethoxylated, polypropoxylated or polyglycerolated and have at least one fatty chain; copolymers of ethylene oxide and/or of propylene oxide; condensates of ethylene oxide and/or of propylene oxide with fatty alcohols; polyethoxylated fatty amides; polyglycerolated fatty amides; ethoxylated fatty acid esters of sorbitan; ethoxylated oils from plant origin; fatty acid esters of sucrose; fatty acid esters of polyethylene glycol; fatty acid mono or diesters of glycerol; (C₆-C₂₄)alkylpolyglycosides; N—(C₆-C₂₄)alkylglucamine derivatives; and amine oxides, and mixtures thereof.

33. The cosmetic composition according to claim 32, wherein the (c) at least one nonionic surfactant with an HLB of greater than 10 is chosen from polyethoxylated fatty alcohols or esters, wherein the number of ethylene oxide units ranges from 7 to 100.

34. The cosmetic composition according to claim 24, wherein the amount of the (c) at least one nonionic surfactant with an HLB of greater than 10 ranges from about 1% to about 30% by weight, relative to the total weight of the composition.

35. The cosmetic composition according to claim 24, wherein the amount of the (d) water ranges from about 10% to about 70% by weight, relative to the total weight of the composition.

36. The cosmetic composition according to claim 24, further comprising (e) at least one alkaline agent.

37. The cosmetic composition according to claim 36, wherein the (e) at least one alkaline agent is chosen from non-volatile alkaline agents.

38. The cosmetic composition according to claim 37, wherein the (e) at least one alkaline agent is chosen from organic amines, inorganic bases, organic amine salts, and ammonium salts, and mixtures thereof.

39. The cosmetic composition according to claim 37, wherein the non-volatile alkaline agent is chosen from inorganic alkaline agents chosen from alkaline metal hydroxides, alkaline earth metal hydroxides, alkaline metal (hydrogeno)carbonates, alkaline earth metal (hydrogeno) carbonates, ammonium (hydrogeno)carbonates, alkaline metal metasilicates, ammonium metasilicates, and mixtures thereof.

40. The cosmetic composition according to claim 37, wherein the non-volatile alkaline agent is chosen from organic alkaline agents chosen from monoamines, derivatives and salts of monoamines, diamines, derivatives and salts of diamines, polyamines, and derivatives and salts of polyamines amino acids and derivatives thereof, oligomers of amino acids and derivatives thereof, polymers of amino acids and derivatives thereof, urea and derivatives thereof, guanidine and derivatives thereof, and mixtures thereof.

41. The cosmetic composition according to claim 37, wherein the non-volatile alkaline agent is chosen from alkanolamines.

42. The cosmetic composition according to claim 24, further comprising (f) at least one oxidative dye.

43. The cosmetic composition according to claim 24, wherein the composition is in the form of a gel.

44. A process of dyeing keratin fibers comprising applying a cosmetic composition to said keratin fibers in the presence of an oxidizing composition comprising at least an oxidizing agent, said composition comprising:
(a) at least one fatty substance;
(b) at least one nonionic surfactant with an HLB of 10 or less;
(c) at least one nonionic surfactant with an HLB of greater than 10; and
(d) water
wherein
the weight ratio of the total amount of the (b) at least one nonionic surfactant with an HLB of 10 or less and the (c) at least one nonionic surfactant with an HLB of greater than 10, to the amount of the (a) at least one fatty substance ranges from 0.6 to 6.0, and
the weight ratio of the total amount of the (a) at least one fatty substance, (b) at least one nonionic surfactant with an HLB of 10 or less, and (c) at least one nonionic surfactant with an HLB greater than 10, to the amount of the (d) water ranges from 0.5 to 1.30.

45. The process according to claim 44, wherein the cosmetic composition and the oxidizing composition are mixed prior to the application onto the keratin fibers.

46. A multi-compartment device comprising, at least:
a first compartment comprising a cosmetic composition, and
a second compartment comprising an oxidizing composition,
wherein said cosmetic composition comprises:
(a) at least one fatty substance;
(b) at least one nonionic surfactant with an HLB of 10 or less;
(c) at least one nonionic surfactant with an HLB of greater than 10; and
(d) water
wherein
the weight ratio of the total amount of the (b) at least one nonionic surfactant with an HLB of 10 or less and the (c) at least one nonionic surfactant with an HLB of greater than 10, to the amount of the (a) at least one fatty substance ranges from 0.6 to 6.0, and
the weight ratio of the total amount of the (a) at least one fatty substance, (b) at least one nonionic surfactant with an HLB of 10 or less, and (c) at least one nonionic surfactant with an HLB greater than 10, to the amount of the (d) water ranges from 0.5 to 1.30.
